(19) United States
(12) Patent Application Publication
(10) Pub. No.: US 2022/0296920 A1
(43) Pub. Date: Sep. 22, 2022

(54) METHOD AND SYSTEM OF SENSING AND ANALYSIS FOR SKIN TREATMENT PROCEDURES

(71) Applicant: LUMENIS BE LTD., YOKNEAM (IL)

(72) Inventors: Andrey Gandman, Haifa (IL); Israel Schuster, Kiryat-Tivon (IL)

(21) Appl. No.: 17/834,059

(22) Filed: Jun. 7, 2022

Related U.S. Application Data
(63) Continuation of application No. 17/565,709, filed on Dec. 30, 2021.
Application No. 17/565,709 is a continuation-in-part of application No. 17/226,235, filed on Apr. 9, 2021.
(60) Provisional application No. 63/132,554, filed on Dec. 31, 2020.

(51) Int. Cl. A61N 5/06; A61N 5/067; G16H 20/40

(52) U.S. Cl. CPC A61N 5/0616; A61N 5/067; G16H 20/40; A61N 2005/0628; A61B 2017/00057

(57) ABSTRACT

Apparatus for analyzing skin tissue. The apparatus comprises an applicator which comprises; one or more sources of illumination light symmetrically surrounding an optical axis of the one or more sources, and one or more sensors configured to obtain measured light along an optical axis of the sensors. The apparatus further comprises a programmable control unit configured to; receive an output of the information sensed of measured light by the sensors, analyze the measured light received from the sensors, provide a list of skin attributes to the display based on analysis of the information sensed of measured light received, and provide a suggested treatment light regimen to a display of the apparatus.

### RELATED APPLICATIONS

This application is a continuation to U.S. non-provisional application Ser. No. 17/565,709 entitled “Method and System for Real Time Monitoring of Cosmetic Laser Aesthetic Skin Treatment Procedures,” which claims the priority of U.S. Provisional Application No. 63/132,554, filed Dec. 31, 2020, and is a Continuation-In-Part to U.S. patent application Ser. No. 17/226,235, filed Sep. 4, 2021, entitled “Real Time Monitoring of Cosmetic Laser Aesthetic Skin Treatment Procedures”, the entire contents of all three of which are herein incorporated by reference and to which the present application's priority is claimed.

### BACKGROUND

Therapeutic and aesthetic energy-based treatments, such as lasers are utilized for procedures on skin, such as hair removal, tattoo removal, vascular removal, pigmented lesions, skin tightening, and/or skin rejuvenation.

Typically, medical personnel manually use a handpiece to deliver such treatments, and the medical personnel will note skin attributes to determine the laser parameters for treatment. The skin attributes may be skin type, presence of tanning, hair color, hair density, hair thickness, blood vessel diameter, blood vessel depth, lesion type, pigment depth, pigment intensity, tattoo color, tattoo type. PCT application number PCT/IL2019/051091, assigned to the assignee of the present disclosure, is directed to some features of the therapeutic and aesthetic energy-based treatment and is herein incorporated by reference in its entirety.

### SUMMARY

In an aspect, an apparatus for treating skin tissue with a source of treatment light comprising: a display; a source for providing treatment light along an optical axis; an applicator. The applicator having a distal end portion comprising: a pathway within the applicator to receive and transmit the treatment light out of the distal end of the applicator along the optical axis; a tip connected to the distal end of the applicator, the tip further comprising one or more sources of illumination light to illuminate the skin tissue; and one or more sensors offset from the optical axis and configured to measure illumination light reflected from the skin tissue.

The apparatus further comprising a programmable control unit, the programmable control unit being configured to; activate the one or more sources of illumination light, such that illumination light is directed to the skin tissue; receive and analyze information sensed from the one or more sensors; generate and provide a list of attributes of the skin based on the analysis of the information sensed of the illumination light reflected from the skin tissue; and generate and provide a suggested treatment light regimen on the display.

In another aspect, the apparatus where the tip comprises a lens for the source of treatment light. The apparatus where one or more sources of illumination light comprises a plurality of light sources symmetrically surrounding the optical axis. Also, the apparatus where the plurality of light sources have different wavelengths of light output and the programmable control unit is configured to select one or more light sources from the plurality of different light source wavelengths and activate the one or more light sources to illuminate the skin tissue.

In a further aspect, the apparatus where the plurality of light sources are LED light sources, and the LED light sources have wavelengths in the range of 300 nm to 1000 nm. The apparatus tip further comprises a substrate for the LED light sources, and the substrate is a printed circuit board for a plurality of LED light sources symmetrically surrounding a pathway of the optical axis, such that the skin tissue is illuminated on the optical axis.

In yet another aspect, the tip is removably connected to the applicator and the tip further comprises pin connections configured to attach and detach the tip from the applicator. The apparatus further comprising image focus optical elements on an image pathway to the one or more sensors.

In another aspect, the one or more sensors are optically placed at a first angle in relation to the optical axis pathway, and the image focus elements are optically placed at a second angle to the optical axis pathway such that a distortion of the illumination light reflected from the skin tissue is corrected.

The tip further comprises a polarization illumination optic element operable to polarize the illumination light from the one or more sources of illumination light. The applicator further comprises a polarization image optic element operable to: polarize illumination light reflected from the skin tissue prior to the one or more sensors receiving the illumination light reflected; and polarize illumination light reflected from the skin tissue in an orthogonal polarization in relation to the polarization illumination optics polarization, such that skin surface layer back scattering of light is avoided.

In one aspect, the applicator further comprises a frame configured to flatten the skin tissue. The source of treatment light is selected from one or more of: a fiber laser source, a solid-state laser source, an Intense Pulse Light (IPL) light source, and a LED light source.

In an aspect, there is a method of treating skin tissue with a source of treatment light comprising: providing a source of treatment light along an optical axis; providing one or more sources of illumination light to illuminate the skin tissue; providing one or more sensors; providing a display; providing a programmable control unit. The method further comprising the programmable control unit: activating the one or more sources of illumination light, such that the illumination light is directed to the skin tissue; processing, by the programmable control unit, the information sensed; displaying on the display, a suggested treatment parameter and a list of skin attributes obtained by processing the information sensed. The method further comprises collecting and storing the information sensed from the one or more sensors. The method even further comprises: providing a plurality of light sources having different wavelengths of light output; selecting, by the programmable control unit, one or more light sources from the plurality of different light source wavelengths; and activating, by the programmable control unit, the one or more light sources to illuminate the skin tissue.

In another aspect, the one or more light sources are one or more LED light sources and further comprising activating selectively, by the control unit, one or more of the one or more LED light sources dependent upon a category of skin tissue treatment. The method further comprises activating, by the programmable control unit, one of the one or more sources of illumination light dependent on a desired depth of light penetration into the skin tissue.

In a further aspect, the method further comprises: reactivating, by the programmable control unit, the one or more sources of illumination light after laser treatment of the skin tissue; and determining, by the programmable control unit, a condition of the skin tissue after treatment of the skin tissue.

Also, the method wherein processing, by the programmable control unit, the information sensed by the one or more sensors, further comprises: analyzing, by the programmable control unit, the information sensed of the one or more sensors; and matching that information to a second set of information. The second set of information is at least one of the following: information contained in a lookup table in a memory associated with the programmable control unit; information contained in one or more embedded algorithms contained in a memory associated with the programmable control unit; or information using artificial intelligence methods and deep learning contained in a memory associated with the programmable control unit. The treatment regimen is then selected outputted onto a display.

In yet a further aspect, the method where the provided one or more sensors are offset from an optical axis of a treatment light, and optical image elements are placed at an angle from the one or more sensors such that a distortion of the illumination light reflected from the skin tissue created by the offset of the one or more sensors is corrected.

In one aspect, the method wherein the list of skin attributed displayed comprises at least one of the following;
i) skin melanin level,
ii) skin melanin map,
iii) skin erythema level,
iv) hair melanin level,
v) hair diameter,
vi) hair density,
vii) hair width,
viii) hair count,
ix) erythema map,
x) tattoo ink analysis mapping and measurement,
xi) wrinkles map,
xii) lesion map,
xiii) acne map,
xiv) cellulite map,
xv) erythema level,
xvi) blood vessel map,
xvii) RGB image,
xviii) blood vessel depth,
xix) blood vessel diameter,
xx) melanin contrast,
xxii) melanin depth,
xxiii) pigment depth, and
xxiv) hair mask file.

In a further aspect, the method further comprises: providing a polarization illumination optic element operable to polarize in a first polarization the illumination light; and providing a polarization image optic element operable to polarize in a second polarization, the reflected illumination light from the skin tissue prior to the one or more sensors receiving the reflected illumination light, wherein the second polarization is orthogonal to the first polarization.

In yet a further aspect, there is a method for determining a skin treatment regimen, the method comprising:
illuminating a skin tissue with a plurality of illumination light beams having respectively a plurality of light wavelengths,
detecting illumination light reflected from the skin tissue and generating image data,
analyzing the image data and generating skin data indicative of skin optical or physical properties up to 5 millimeters deep, and
analyzing the skin data to determine the skin treatment regimen.

In an aspect, there is provided an apparatus for analyzing skin tissue, comprising,
an applicator, the applicator having a distal end portion comprising,
   a tip connected to the distal end of the applicator, the tip further comprising one or more sources of illumination light to illuminate the skin tissue,
   a pathway within the applicator to transmit the illumination light from the sources of illumination light to the skin tissue; and
   one or more sensors configured to detect and measure illumination light reflected from the skin tissue, wherein the pathway further transmits the reflected light from the skin tissue to the one or more sensors;
a programmable control unit, the programmable control unit being configured to:
   activate the one or more sources of illumination light, such that illumination light is directed to the skin tissue,
   receive and analyze information sensed from the one or more sensors,
   generate and provide a list of attributes of the skin based on the analysis of the information sensed of the illumination light reflected from the skin tissue; and
   generate a suggested treatment light regimen; and
a display, configured to display the light treatment regimen.

In another aspect, the programmable control unit is configured to employ artificial intelligence and deep learning methods to perform the analysis.

In yet another aspect, the one or more sources of illumination light comprise a plurality of light sources symmetrically surrounding an optical axis of the one or more sources.

In a further aspect, the plurality of light sources has a plurality of different wavelengths of light output, and wherein the programmable control unit is configured to select one or more light sources from the plurality of different light source wavelengths and activate the one or more light sources to illuminate the skin tissue.

In another aspect, the plurality of light sources are LED light sources having wavelengths in the range of 300 nm to 1000 nm.

In a further aspect, the tip further comprises a substrate for the LED light sources, the substrate comprising a printed circuit board (PCB) mounting the LED light sources symmetrically surrounding an aperture of the PCB mounting, such that the skin tissue illumination is centered about the optical axis of the sources.

In another aspect, the tip is removably connected to the applicator.

In yet another aspect, the apparatus further comprises image focus optical elements on an optical axis of the one or more sensors.

In another aspect, the tip further comprises at least one first polarizer disposed in front of and coaxial with an optical axis of the sources, configured to polarize the illumination light from the one or more sources of illumination light at a first polarization; and the applicator further comprises at least one second polarizer disposed in front of and coaxial with an optical axis of the one or more sensors, configured to polarize the illumination light reflected from the skin tissue to the one or more sensors in a second polarization, wherein the second polarization is orthogonal to the first polarization.

In yet another aspect, the applicator further comprises a frame configured to flatten the skin tissue when the applicator is brought into contact with the skin tissue.

In an aspect, there is provided a method for analyzing skin tissue, the method comprising,
providing one or more sources of illumination light to illuminate the skin tissue;
providing one or more sensors;
providing a display;
providing a programmable control unit;
activating, by the programmable control unit, the one or more sources of illumination light, such that the illumination light is directed to the skin tissue;
collecting light reflected from the skin by the one or more sensors in response to the illumination light;
processing, by the programmable control unit, the illumination light received by the one or more sensors and generating data sensed; and
displaying, by the programmable control unit, on the display, a suggested treatment parameter and a list of skin attributes obtained by processing the data sensed.

In another aspect, the method further comprises storing, by the programmable control unit, the data sensed.

In yet another aspect, the method further comprises,
providing a plurality of sources of illumination light having different wavelengths of light output;
selecting, by the programmable control unit, one or more light sources from the plurality of different light source wavelengths; and
activating, by the programmable control unit, the one or more light sources to illuminate the skin tissue.

In another aspect, the one or more light sources of illumination light are one or more LED light sources and the method further comprises activating selectively, by the programmable control unit, one or more of the one or more LED light sources dependent upon at least one of the following: a category of skin tissue treatment, and a desired depth of light penetration into the skin tissue.

In yet another aspect, the method further comprises,
reactivating, by the programmable control unit, the one or more sources of illumination light after treatment of the skin tissue by a source of treatment light; and
determining, by the programmable control unit, a condition of the skin tissue after the treatment of the skin tissue.

In yet another aspect, the list of skin attributes displayed comprises at least one of the following:
i) skin melanin level, ii) skin melanin map, iii) skin erythema level, iv) hair melanin level, v) hair diameter, vi) hair density, vii) hair width, viii) hair count, ix) erythema map, x) tattoo ink analysis mapping and measurement, xi) wrinkles map, xii) lesion map, xiii) acne map, xiv) cellulite map, xv) erythema level, xvi) blood vessel map, xvii) RGB image, xviii) blood vessel depth, xix) blood vessel diameter, xx) melanin contrast, xxi) melanin depth, xxii) pigment depth, and xxiii) hair mask file.

In another aspect, the method further comprises,
providing at least one first polarizer element disposed in front of and coaxial with an optical axis of the sources, configured to polarize the illumination light from the one or more sources of illumination light at a first polarization; and
providing at least one second polarizer element disposed in front of and coaxial with an optical axis of the one or more sensors, configured to polarize the illumination light reflected from the skin tissue to the one or more sensors in a second polarization, wherein the second polarization is orthogonal to the first polarization.

In an aspect, there is provided a method for determining a skin treatment regimen, the method comprising:
illuminating a skin tissue with a plurality of illumination light beams having respectively a plurality of light wavelengths;
detecting illumination light reflected from the skin tissue and generating image data;
analyzing the image data and generating skin data indicative of skin optical or physical properties up to 5 millimeters deep; and
analyzing the skin data to determine the skin treatment regimen.

### BRIEF DESCRIPTION OF THE DRAWINGS

Some embodiments of apparatuses and/or methods will be described in the following drawings, by way of example only.

FIG. 1 illustrates a high-level functional architecture scheme of the present disclosure.

FIGS. 2 and 3 illustrate schematics of an applicator which embody aspects of the present disclosure.

FIGS. 4A and 4B illustrate schematics of an applicator, in some embodiments of the present disclosure.

FIG. 5 illustrates an illumination element according to some embodiments of the present disclosure.

FIG. 6A-I illustrate a smart tip according to some embodiments of the present disclosure.

FIG. 7 illustrates an imaging unit on an applicator according to some embodiments of the present disclosure.

FIG. 8 illustrates an imaging unit on an applicator according to some embodiments of the present disclosure.

FIG. 9 illustrates a flow chart of a method according to some embodiments of the present disclosure.

FIG. 10A illustrates the histological layers of typical human skin tissue.

FIG. 10B illustrates a schematic representation of various layers of human skin tissue.

FIGS. 11A and 11B are two series of skin tissue images obtained according to some embodiments of the present disclosure.

### DETAILED DESCRIPTION

The present invention is directed to provide a system and method to provide dynamic imaging and real time monitoring of laser treatments in a laser treatment system. A treatment laser may be one that targets the skin tissue, gets absorbed by one or more chromophores and causes a cascade of reactions, including photochemical, photothermal, thermal, photoacoustic, acoustic, healing, ablation, coagulation, biological, tightening or other any other physiological effect. Those reactions create the desired treatment outcomes such as permanent hair removal, hair growth, pigmented or vascular lesion treatment of soft tissue, rejuvenation or tightening, acne treatment, cellulite treatment, vein collapse, or tattoo removal which may include mechanical breakdown of tattoo pigments and crusting.

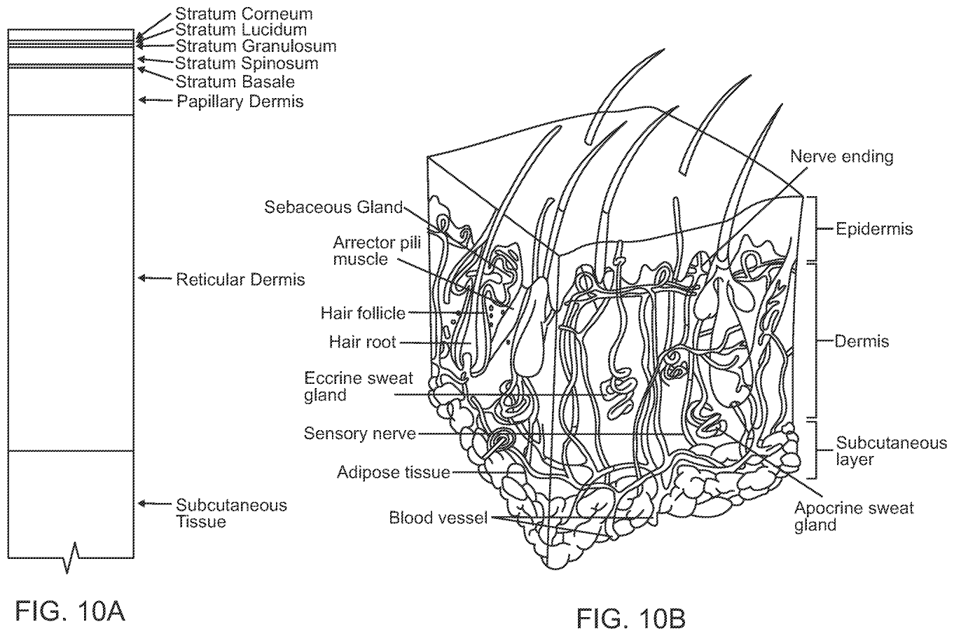

Skin tissue is a very complex biological organ. Although the basic structure is common to all humans (see FIGS. 10A and 10B), there are many variations within the different areas in a specific individual and among individuals. Variations include skin color (melanin content in Basal layer), hair color and thickness, collagen integrity, blood vessel structure, vascular and pigmented lesions of various types, foreign objects like tattoos, etc.

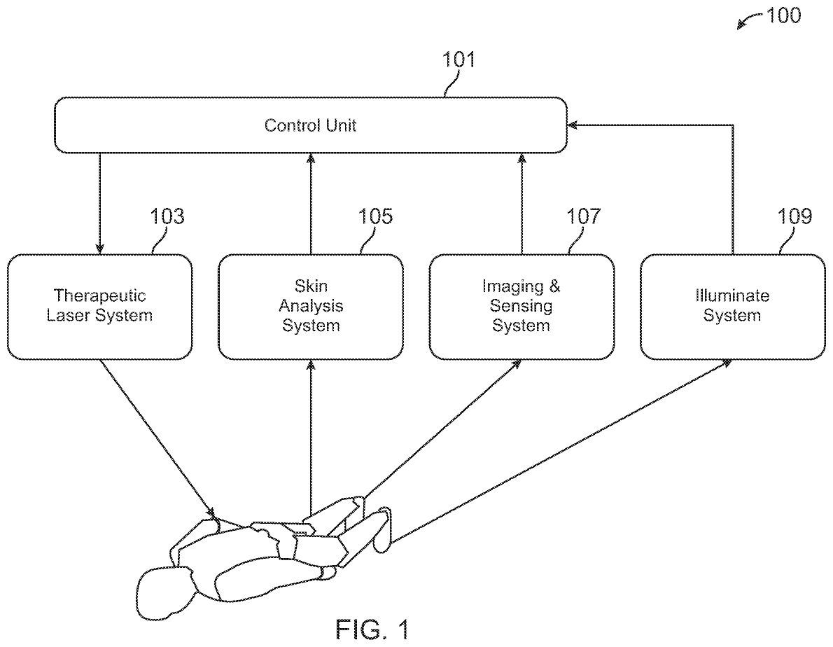

FIG. 1 is a conceptual illustration of a high-level system functional architecture of a diagnostic and treatment system 100 for skin. A programmable control unit 101 manages a therapeutic laser system 103, skin analysis and diagnostic system 105, a sensing system 107 and an illumination system 109. In some embodiments, the therapeutic laser system 103, is a therapeutic energy-based system and that energy-based system may be Intense Pulsed Light (IPL) or Radio Frequency (RF) or a combination of both IPL and RF.

In some embodiments, diagnostic and treatment system 100 illuminates a target skin or tissue in various wavelengths and sensing system 107 captures the illumination light reflected or back scattered from skin tissue. The sensors measure the light reflected or back scattered from the illuminated skin tissue (hereinafter images) thus obtaining information. These images (different wavelengths, polarizations, and patterns) with their corresponding meta-data for each wavelength illuminated are thereby obtained.

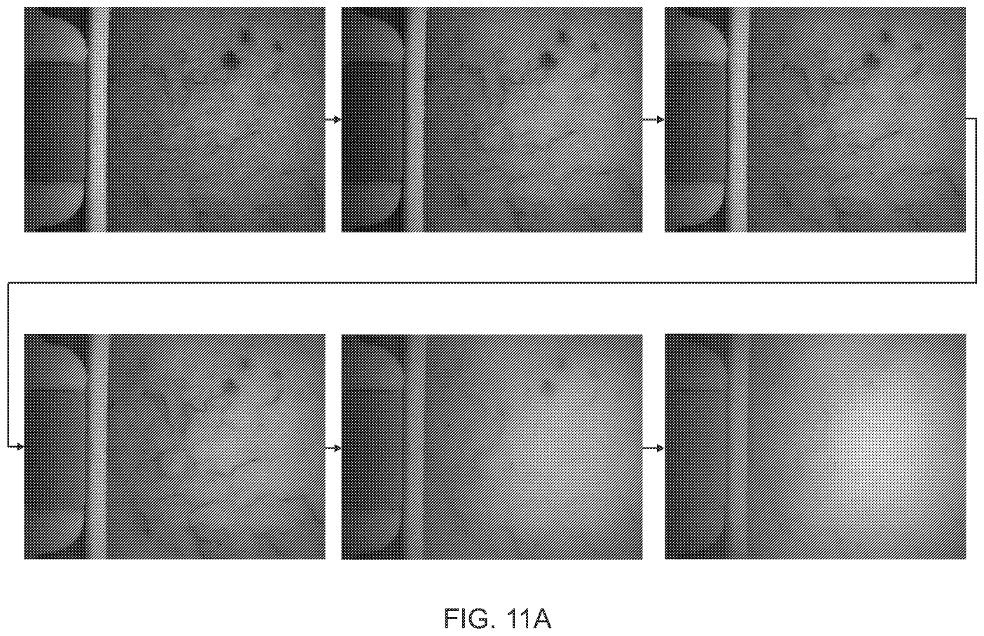

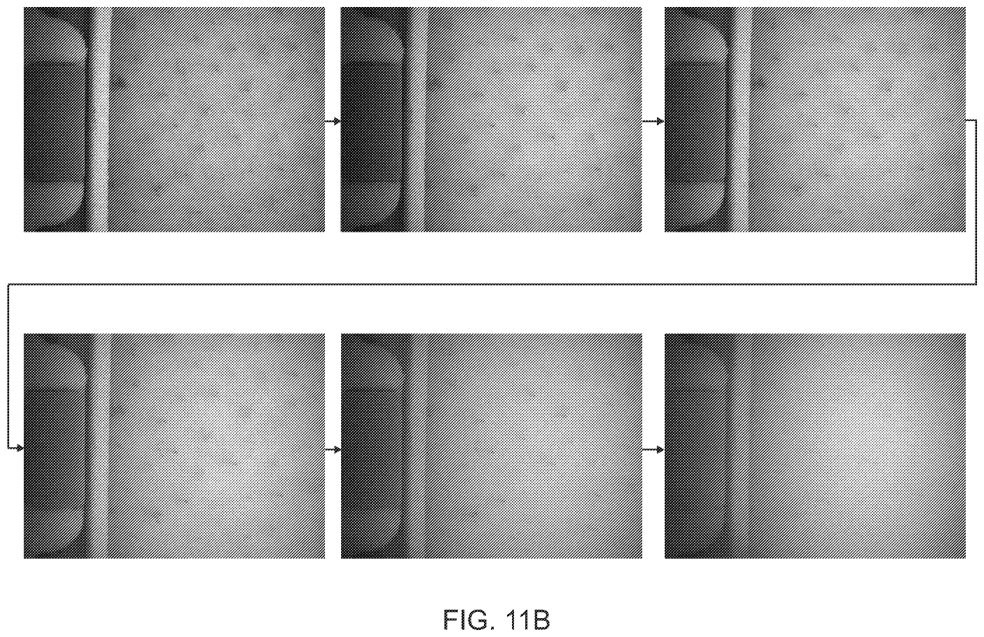

In some embodiments, images and corresponding meta-data (hereinafter diagnostic data) are parsed and analyzed for more information about the target tissue and/or its location. With this method, basic skin optical and physical properties up to about 5 millimeters deep may be obtained (see FIGS. 11A and 11B.) The diagnostic data may be analyzed by, and is not limited to, the following; Principal Component Analysis (hereinafter PCA), physical modelling, unique algorithm, neural network algorithms, or any combination thereof. In some embodiments, the diagnostic data is collected and stored into a database. In some embodiments, the parsed and analyzed diagnostic data are also collected and stored into the database.

In some embodiments, the PCA is the method of analysis and the PCA enables robust classification of valuable parameters while reducing overall dimensionality of the acquired data. The most relevant parameters may be employed for the development of a physical laser-tissue interaction model, including, for example, thermal relaxation and soft tissue coagulation. Moreover, large amounts of highly correlated data allow for construction of empirical equations which are based on quantitative immediate biological responses like erythema in hair removal and frosting formation in tattoo removal treatments.

In some embodiments, use of artificial intelligence technology e.g. deep learning (DP) may be used to analyze the diagnostic data. Deep learning involves the use of complex, multi-level “deep” neural networks to create systems that can perform feature detection from massive amounts of unlabeled training data.

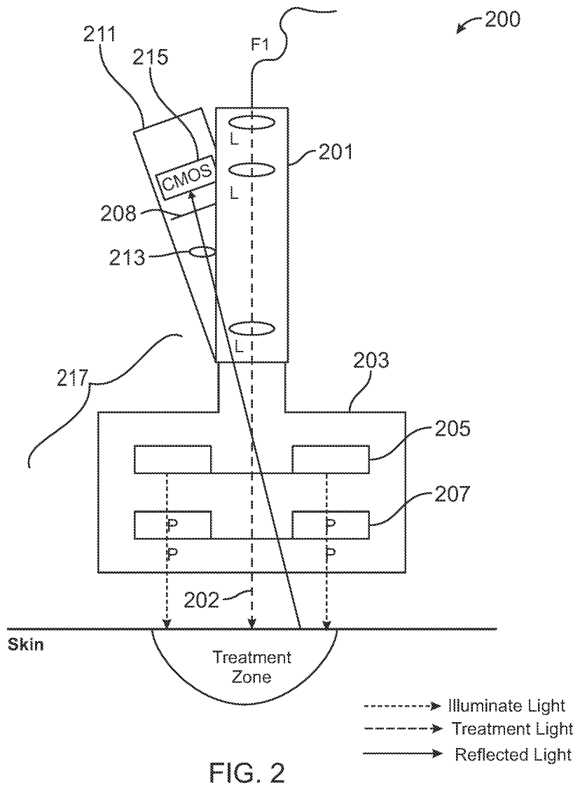

In some embodiments of the diagnostic and treatment system, an integrated treatment and imaging laser handheld applicator (hereinafter applicator) is operable to collect data from a target tissue. In some embodiments, the applicator does not directly contact the skin. In some embodiments, the applicator directly contacts the skin. FIG. 2 is a functional diagram of an exemplary embodiment of an applicator 200, and many other variations of an applicator 200 may be implemented. A treatment laser unit 201 comprises lenses L and other optic features as may be required. These optic features will vary with clinical indications and the effect of coupling the applicator's treatment laser unit 201 with the diagnostic and treatment laser system 103. The treatment laser unit 201 may further comprises a high-power laser fiber input source (F1),

Treatment laser unit 201 may be a laser delivery unit. In some embodiments, the treatment laser unit is an applicator which is connected to a laser console with a fiber and/or an articulated arm. In some embodiments, the treatment laser unit may have an integrated laser or light source housed within. In the current disclosure, the laser may be in the Splendor X system available from Lumenis Ltd. of Israel, and the treatment laser unit may be part of the applicator that delivers the laser to the target tissue. The treatment laser unit and the treatment laser system have different parameters of use that include wavelength, spot size, fluence, pulse duration, and pulse rate.

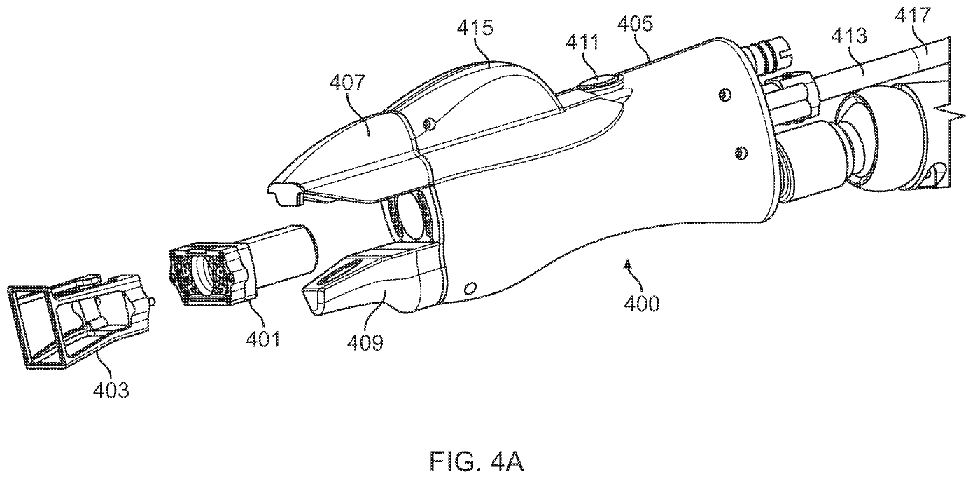

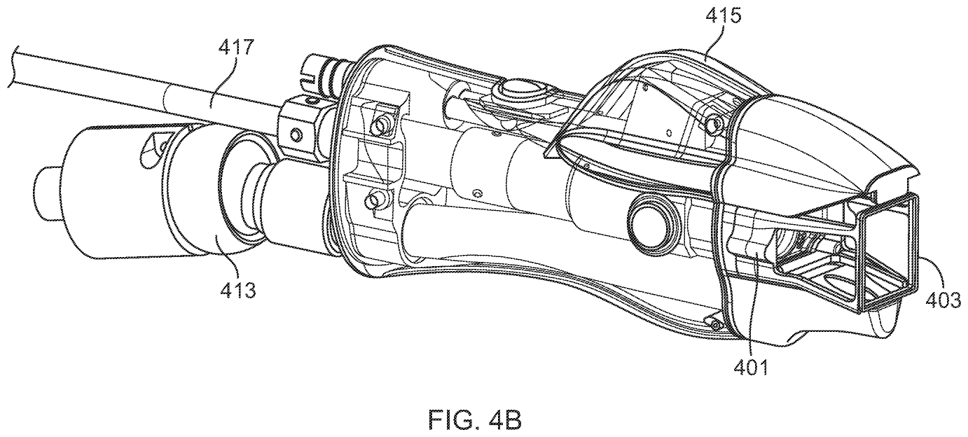

An illumination unit 203, in some embodiments, comprises illumination substrate 205 to support specific illumination elements, polarization illumination optics 207, and clear protection element (not shown). In some embodiments, this illumination unit may have various optics and physical configurations. Optical axis 202 of laser system 201 is barrier free on the path to the skin, and the illumination unit optics may be configured such that there is no barrier to the optical axis. In some embodiments, the illumination elements are a configuration of intense light such as Light Emitting Diodes (hereinafter LED light source.) The illumination system may be housed in a tip component 217 (401 in FIG. 4B) further discussed below.

In some embodiments, applicator 200, further comprises an image unit 211 for obtaining images. In some embodiments, the image unit has a camera lens 213, polarization image optics 208 and a CMOS or other sensor 215. In some embodiments, polarization image optics 208 have polarization orthogonal to the polarization illumination optics 207, such that skin surface layer back scattering of the same illumination polarization is avoided.

In some embodiments, image unit 211 may have folding mirrors (FM) or other optic elements required to ensure accurate capture by sensor 215 of images based on the position of the image unit on the applicator 200. In some embodiments, the programmable control unit 101 prevents the sensor from capturing images during operation of the laser system. In some embodiments, the image unit is protected by a shutter.

In some embodiments of the current disclosure, the system may be a diagnostic system and not a treatment system. In such embodiments, an applicator may have an illumination unit and an imaging unit (not shown) with connection to a skin analysis and diagnostic system 105.

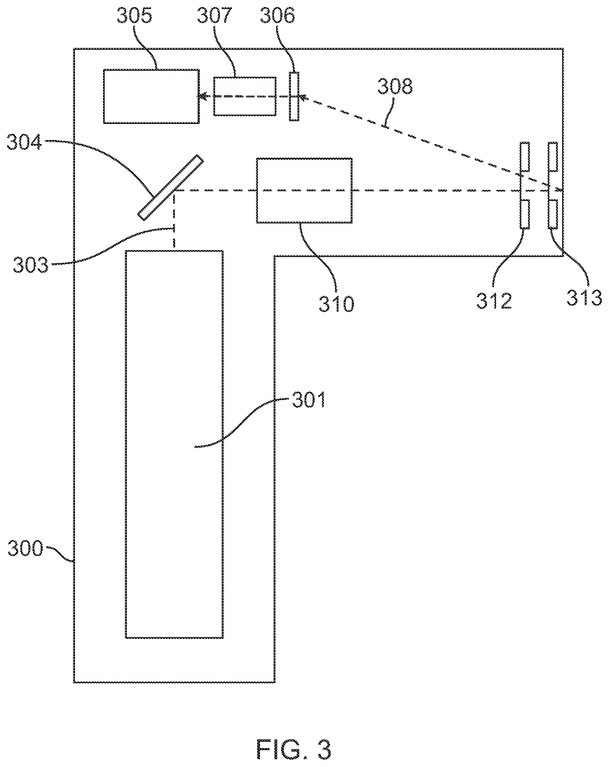

In some embodiments of an applicator, the laser power source may be a laser module 301 included in the applicator as illustrated FIG. 3. Here, instead of the laser input source (F1), there may be a laser module 301, which may be a solid-state laser source of a known type. Applicator 300 may further comprise a folding mirror 304 to alter a laser axis path 303. Further down the laser optical path, in this example, are focus optics 310, an illumination substrate 312 and a polarization illumination film or optics 313. In some embodiments, the imaging unit of applicator 300 comprises a sensor 305, polarization image optics 307, and focus optics 306. An imaging axis 308 is the path of the image to the imaging unit. In some embodiments, the angle of focus optics 306 and sensor 305 are optically arranged such that the image provided is a flat image or perpendicular to the laser axis 303 and not the imaging axis 308.

In some embodiments, an applicator 400 has a handle 405, a tip 401 that houses an illumination unit that attaches to handle 405, as illustrated in FIGS. 4A-4B. In some embodiments, a frame 403 is configured to stretch or flatten a target tissue for obtaining images. In some embodiments, frame 403 connects to tip 401 with magnets or similar connections known in the art. In some embodiments, the frame stretches or flattens a skin treatment area to 0-2 mm to allow using an imaging unit with constant focus.

The applicator 400 may have a suction channel 407 for receiving skin debris produced by a treatment laser, as well as a skin cooling unit 409. In some embodiments, a switch 411 is operable for a user to start the process of obtaining images from the target tissue. The handle may have an imaging unit housed in area 415 of the applicator 400. Treatment laser umbilical 417 and coolant hose 413 are configured to connect applicator 400 to a base diagnostic and treatment system or console.

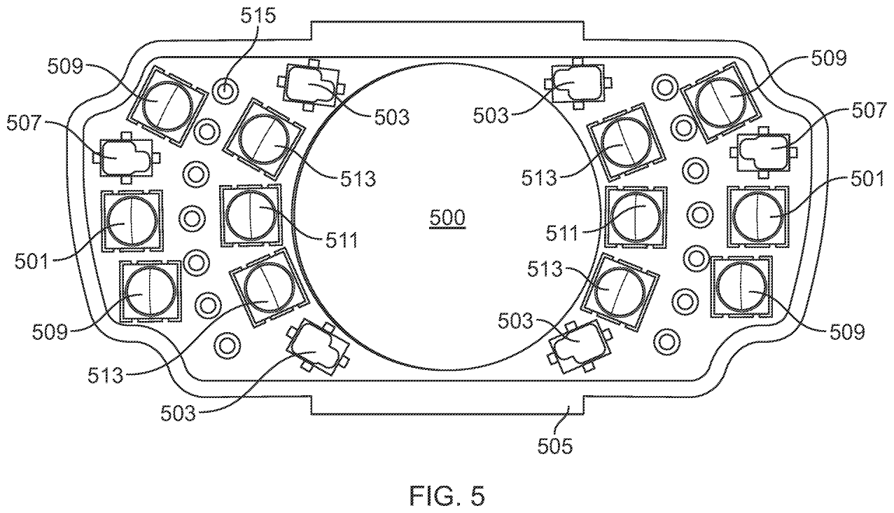

FIG. 5 is an illustration of an illumination substrate 505 that may be housed in a tip 401. In some embodiments, substrate 505 or the illumination unit may be housed directly in the applicator, and not in a tip. By way of specific example, the illumination substrate 505 may be a printed circuit board (hereinafter PCB) in accordance with one or more embodiments of the present disclosure. The PCB comprises a plurality of LED light sources having different wavelengths. The LED light sources may be positioned symmetrically around the laser optical path 500. In some embodiments, LED light sources have wavelengths in the range of 300 nm to 1100 nm.

In the specific example of FIG. 5, there are two red LED light sources 501 with a wavelength of 660 nm. Four yellow LED light sources 503 with a wavelength of 590 nm. Two infrared LED light sources 507 with a wavelength of 860 nm. Four cyan LED light sources 509 with a wavelength of 490 nm. Two blue LED light sources 511 with a wavelength of 450 nm. Four green LED light sources 513 with a wavelength of 530 nm. In some embodiments, the PCB further comprises pins 515 for connection to the system and applicator. A memory chip (not shown) may be placed on the opposite side of the PCB and is configured to identify to an applicator a tip type that is connected. The number of LED light sources for each wavelength may be determined by the intensity of the wavelength required to obtain an image illuminated evenly.

By way of example, FIG. 11A illustrates one series of skin images of a target tissue, each acquired with a different illumination wavelength, obtained by the current disclosure's device and method. FIG. 11B is a second series of images, of a different target tissue, again acquired with a different illumination wavelength and obtained by the current disclosure's device and method. The various levels of melanin, epidermal and dermal thickness and blood content of a target tissue is exposed with respect to the different light wavelengths. Basic skin optical and physical properties up to about 5 millimeters deep may be obtained and mapped spatially and across depth.

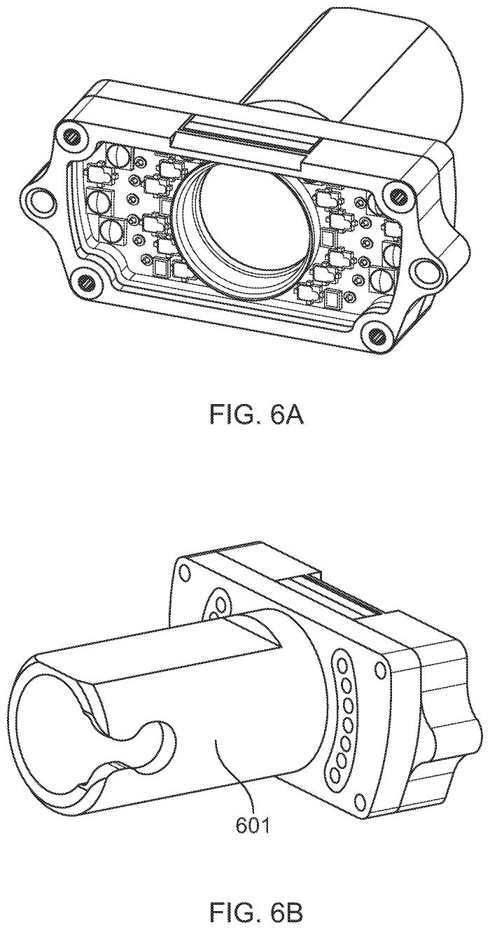

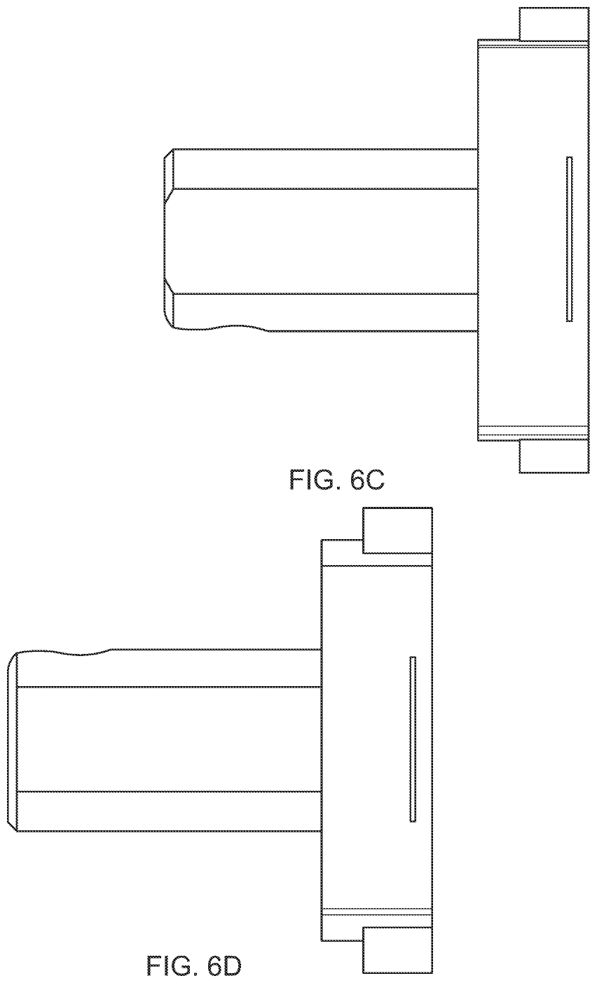

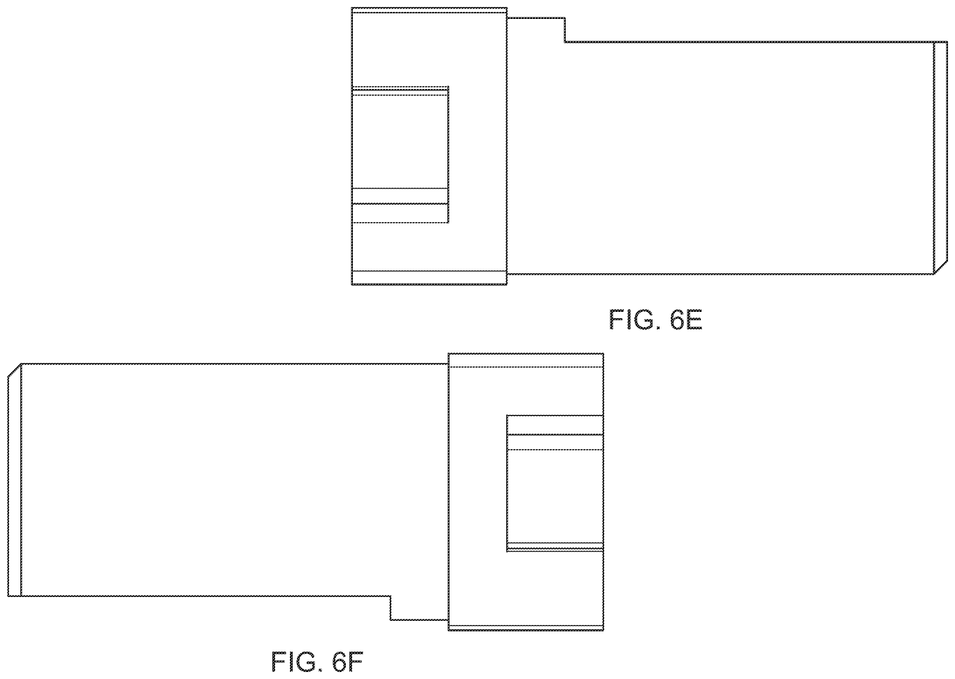

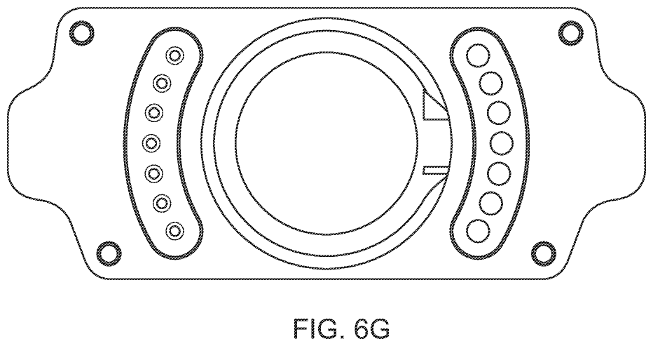

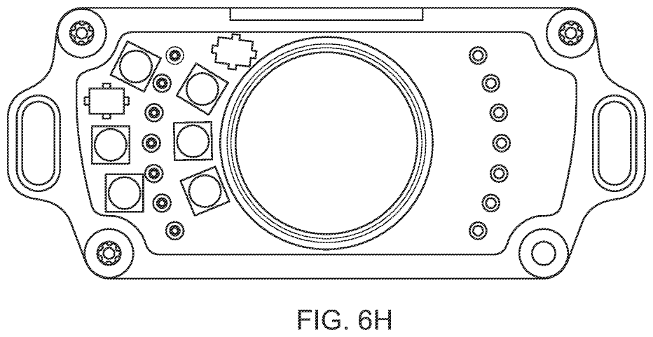

In some embodiments, the lens optics of the laser are housed in the tip. FIG. 6A to FIG. 6I illustrate a smart tip in accordance with one or more embodiments the current disclosure. Tip 401 may be removably attached to applicator 400. In this example, tip 401 comprises; a tip base 600, a laser path lens 601, laser lens holder 603, illumination substrate or LED PCB 505, polarization illumination optics 605, a spacer 607, a window 609 that protects and seals the LED PCB 505, window housing 610 and a connection method 611 of any known type. The polarization illumination optics of the tip polarize the LED light sources and comprises a barrier free area in the center for the laser treatment to travel through.

Cooling unit 409, in some embodiments, may lower the temperature of the LED light sources to between 0 to 5 degrees Celsius. In some embodiments, tip 401 comprises a heating system (not shown) configured to maintain the temperature of the LED light sources in the range of 25 to 35 degrees Celsius, which is optimal to maintain the intensity of the LED light sources. In some embodiments, an algorithm for analysis will include a correction for any lower intensity of the LED light sources when there is no heating system.

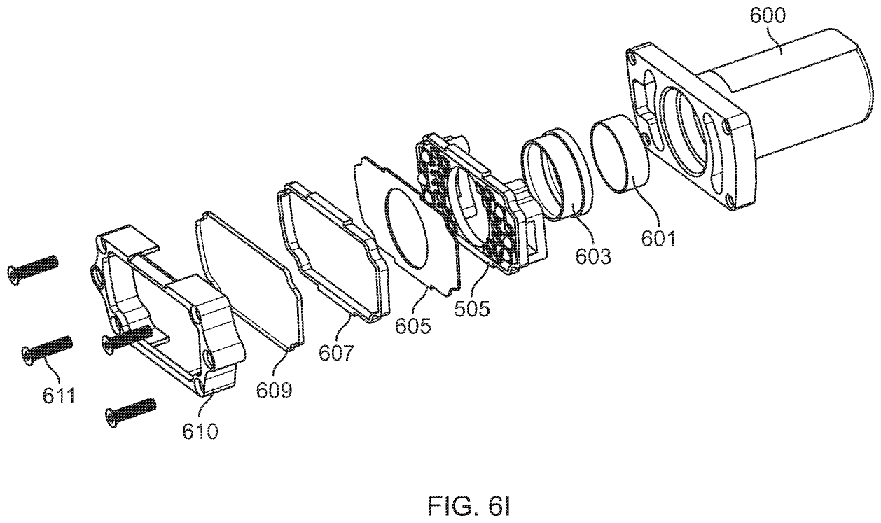

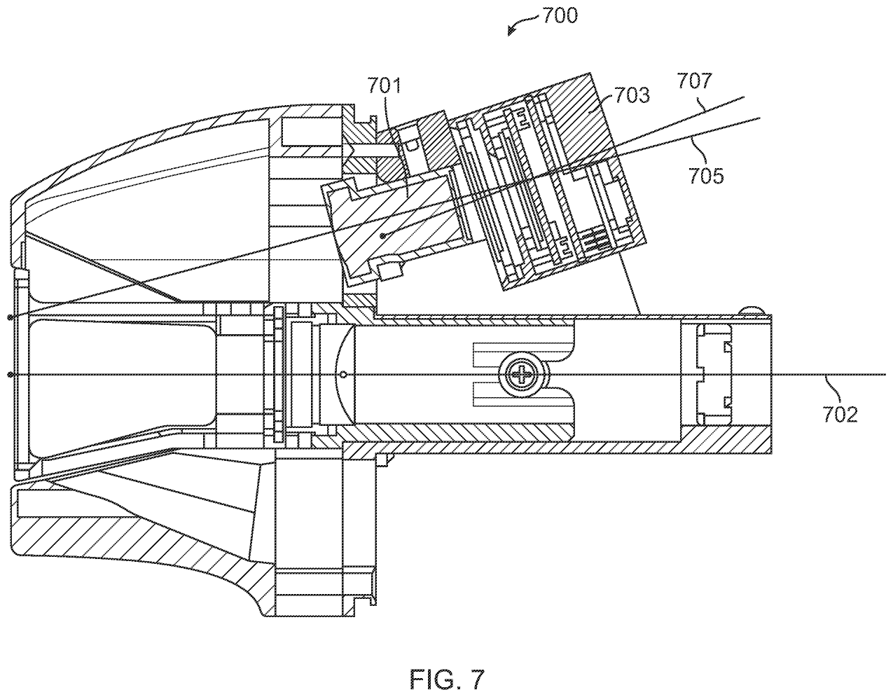

FIG. 7 illustrate an imaging unit 700 that may be housed in applicator 400 in the imaging housing 415. In this example of an imaging unit, the optical axis angle 705 of lens 701 and the optical axis 707 of sensor 703 are offset and arranged such that the image obtained corrects a probable distortion based on the offset sensor 703. The angled position of sensor 703 relative to the main optical axis of the laser 702 may be configured to share the field of view of the sensor and treatment area that may be covered by the laser. Since laser axis 702 is perpendicular to the target tissue, an angled sensor 703 results in a distorted image. Countered angled lens 701 is configured to compensate and correct such distortion. In this specific example, the lens is positioned such that the lens axis 705 is a 14-degree angle to the laser axis 702 and the sensor axis 707 is positioned in a 4.30-degree angle to the lens axis 705.

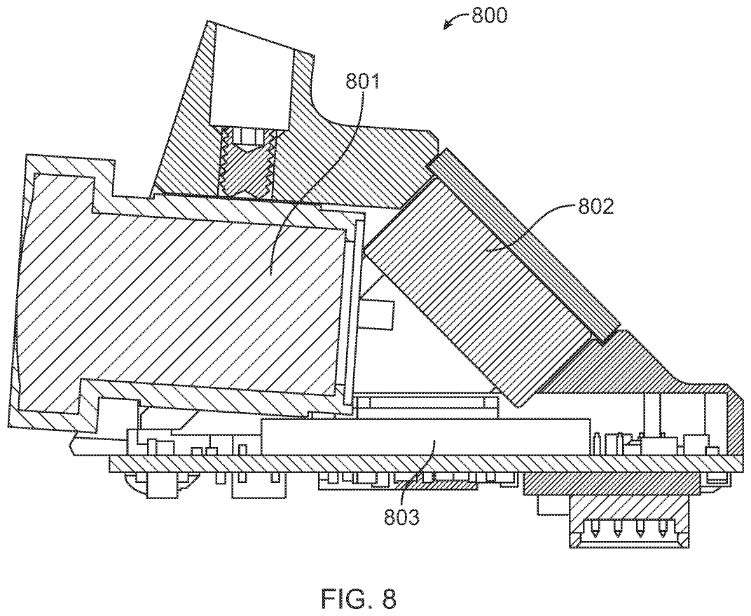

FIG. 8 illustrates, in some embodiments, an imaging unit 800 that may be housed in imaging housing 415. In this configuration, imaging lens 801 has a lens axis (not shown) to a target tissue and that lens axis path is folded by a folding image mirror 802, or similar optical element known in the art, to direct the image to sensor 803. In this example, the laser axis 702 is still perpendicular to the target tissue, and the sensor placement alone will result in a distorted image of the target tissue. The optical arrangement of lens 801, the folding mirror 802 and sensor 803 are all configured to compensate and correct for such distortion. In some embodiments, the correction of a distortion based on sensor placement is done with a computer algorithm.

The programmable control unit of diagnostic and treatment system may be housed within a laser console and may comprise a suitable processor or computing unit. In some embodiments, the computing unit may comprise one or more processors and instruction stored on non-transitory computer-readable medium, which may be read and executed by the processor or processors.

In some embodiments, the programmable control unit is configured to acquire and analyze the diagnostic data. The programmable control unit may be further configured to manage the following components: the sensor of the image system, the LED light sources of the illumination system, and the laser of the laser system.

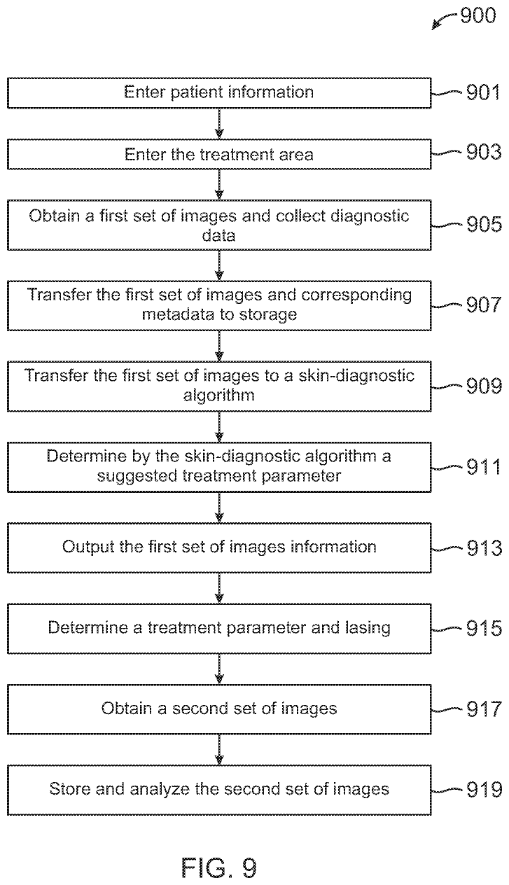

FIG. 9 illustrates an example of a flowchart of method 900, in accordance with one or more embodiments of the present disclosure. Method 900 may include a user entering 901 a patient's information and entering 903 the treatment area into an input for a diagnostic and treatment system.

Method 900 may include collecting 905 diagnostic data by obtaining a first set of images of a target tissue. In some embodiments, a user will press a start button 411 to obtain the first set of images. In some embodiments, this data collection is done dynamically in real time before a laser treatment.

Method 900 may include transferring 907 the first set of images and their corresponding metadata to a database storage system or device. The metadata may include the first set of image's illumination wavelength, LED brightness, camera exposure, and camera gain

Method 900 may include transferring 909 a first set of images to a skin-diagnostic algorithm to analyze the diagnostic data.

Method 900 may include the skin-diagnostic algorithm determining 911 suggested treatment parameters, also known as treatment light regimens, for the target tissue. In some embodiments, the skin-diagnostic algorithm may use diagnostic data that may have been previously stored in the data base to assist in analyzing the first set of images. In some embodiments, the laser treatment parameters are set for the diagnostic and treatment system.

Method 900 may include a display unit to output 913 suggested treatment parameters and skin attributes about the first set of images after analysis. The display of skin attributes may include, among other things: skin melanin level, skin melanin map, skin erythema level or map, hair melanin level, hair diameter, hair density, hair width, hair count, and hair mask file. The output information may be in the form of a GUI on the display unit. This display of output information allows for a medical professional to evaluate and determine the parameter of treatment.

Method 900 may include a user determining a treatment parameter and lasing 915 the target tissue.

Method 900 may include obtaining 917 an automatic second set of images of the target tissue after lasing is completed.

Method 900 may include storing and analyzing 919 the second set of images. In some embodiments, this data collection is done dynamically in real time after a laser treatment.

In some embodiments, of the present exemplary method, the skin diagnostic system may have two working modes; an analysis mode for capturing, analyzing and suggesting preset without laser treatment and a treatment mode for capturing before and after image series of the treatment for data collection and analysis. In some embodiments, the skin analysis and diagnostic system 105 may have only an analysis mode for capturing, analyzing, providing relevant data on a display and suggesting presets for treatment.

In some embodiments, the skin and diagnostic system collects data from any input method and may include the skin-diagnostic algorithm to determine suggested treatment parameters, also known as treatment light regimens, (such as peak energy, energy fluence, pulse width, temporal profile, spot size, wavelength, train of pulses, and others), for the target tissue. In some embodiments, the skin-diagnostic algorithm may use diagnostic data that may have been previously stored in the data base to assist in analyzing the data from any input method.

In some embodiments, a display unit outputs suggested treatment parameters and/or skin attributes after analysis of any input method of collecting data. The display of skin attributes may include; skin melanin level, skin melanin map, skin erythema level, hair melanin level, hair diameter, hair density, hair width, hair count, and hair mask file. The output information may be in the form of a GUI on the display unit. This display of output information allows for a medical professional to evaluate and determine the parameter of treatment.

The proposed technology may well provide significant benefits over present commercial devices because none appear to propose an applicator with an angled imaging unit positioned correcting obtained image with optical elements.

A computer, processor or computer system, as used herein, include any combination of hardware and software. A machine-readable medium, as used herein, may include any medium and/or mechanism for storing or transmitting information in a form readable by a machine (e.g., a computing device).

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described herein, various embodiments may be readily combined, without departing from the scope or spirit of the present disclosure.

What we claim is:

1. An apparatus for analyzing skin tissue, comprising,
an applicator, the applicator having a distal end portion comprising,
a tip connected to the distal end of the applicator, the tip further comprising one or more sources of illumination light to illuminate the skin tissue,
a pathway within the applicator to transmit the illumination light from the sources of illumination light to the skin tissue; and
one or more sensors configured to detect and measure illumination light reflected from the skin tissue, wherein the pathway further transmits the reflected light from the skin tissue to the one or more sensors;
a programmable control unit, the programmable control unit being configured to:
activate the one or more sources of illumination light, such that illumination light is directed to the skin tissue,
receive and analyze information sensed from the one or more sensors,
generate and provide a list of attributes of the skin based on the analysis of the information sensed of the illumination light reflected from the skin tissue; and
generate a suggested treatment light regimen; and
a display, configured to display the light treatment regimen.

2. The apparatus of claim 1, wherein the programmable control unit is configured to employ artificial intelligence and deep learning methods to perform the analysis.

3. The apparatus of claim 1, wherein the one or more sources of illumination light comprise a plurality of light sources symmetrically surrounding an optical axis of the one or more sources.

4. The apparatus of claim 3, wherein the plurality of light sources has a plurality of different wavelengths of light output, and wherein the programmable control unit is configured to select one or more light sources from the plurality of different light source wavelengths and activate the one or more light sources to illuminate the skin tissue.

5. The apparatus of claim 3, wherein the plurality of light sources are LED light sources having wavelengths in the range of 300 nm to 1000 nm.

6. The apparatus of claim 5, wherein the tip further comprises a substrate for the LED light sources, the substrate comprising a printed circuit board (PCB) mounting the LED light sources symmetrically surrounding an aperture of the PCB mounting, such that the skin tissue illumination is centered about the optical axis of the sources.

7. The apparatus of claim 1, wherein the tip is removably connected to the applicator.

8. The apparatus of claim 1, further comprising image focus optical elements on an optical axis of the one or more sensors.

9. The apparatus of claim 1, wherein the tip further comprises at least one first polarizer disposed in front of and coaxial with an optical axis of the sources, configured to polarize the illumination light from the one or more sources of illumination light at a first polarization; and the applicator further comprises at least one second polarizer disposed in front of and coaxial with an optical axis of the one or more sensors, configured to polarize the illumination light reflected from the skin tissue to the one or more sensors in a second polarization, wherein the second polarization is orthogonal to the first polarization.

10. The apparatus of claim 1, wherein the applicator further comprises a frame configured to flatten the skin tissue when the applicator is brought into contact with the skin tissue.

11. A method of analyzing skin tissue, the method comprising:
providing one or more sources of illumination light to illuminate the skin tissue;
providing one or more sensors;
providing a display;
providing a programmable control unit;
activating, by the programmable control unit, the one or more sources of illumination light, such that the illumination light is directed to the skin tissue;
collecting light reflected from the skin by the one or more sensors in response to the illumination light;
processing, by the programmable control unit, the illumination light received by the one or more sensors and generating data sensed; and
displaying, by the programmable control unit, on the display, a suggested treatment parameter and a list of skin attributes obtained by processing the data sensed.

12. The method of claim 11, wherein processing the data sensed, by the programmable control unit, further comprises,
analyzing, by the programmable control unit, the data sensed, and
matching, by the programmable control unit, the data sensed to information using artificial intelligence methods and deep learning contained in a memory associated with the programmable control unit;
selecting, by the programmable control unit, a treatment regimen based on the match; and
displaying, by the programmable control unit, the treatment regimen selected on a display.

13. The method of claim 11, wherein processing the data sensed, by the programmable control unit, further comprises,
analyzing, by the programmable control unit, the data sensed, and
matching, by the programmable control unit, the data sensed to at least one of the following:
information contained in a lookup table in a memory associated with the programmable control unit; and
information contained in one or more embedded algorithms contained in a memory associated with the programmable control unit;
selecting, by the programmable control unit, a treatment regimen based on the matching; and
displaying, by the programmable control unit, the treatment regimen selected on a display.

14. The method of claim 11, further comprising storing, by the programmable control unit, the data sensed.

15. The method of claim 11, further comprising;
providing a plurality of sources of illumination light having different wavelengths of light output;
selecting, by the programmable control unit, one or more light sources from the plurality of different light source wavelengths; and
activating, by the programmable control unit, the one or more light sources to illuminate the skin tissue.

16. The method of claim 11, wherein the one or more light sources of illumination light are one or more LED light sources and the method further comprising activating selectively, by the programmable control unit, one or more of the one or more LED light sources dependent upon at least one of the following: a category of skin tissue treatment, and a desired depth of light penetration into the skin tissue.

17. The method of claim 11, further comprising:
reactivating, by the programmable control unit, the one or more sources of illumination light after treatment of the skin tissue by a source of treatment light; and
determining, by the programmable control unit, a condition of the skin tissue after the treatment of the skin tissue.

18. The method of claim 11, wherein the list of skin attributes displayed comprises at least one of the following:
i) skin melanin level, ii) skin melanin map, iii) skin erythema level, iv) hair melanin level, v) hair diameter, vi) hair density, vii) hair width, viii) hair count, ix) erythema map, x) tattoo ink analysis mapping and measurement, xi) wrinkles map, xii) lesion map, xiii) acne map, xiv) cellulite map, xv) erythema level, xvi) blood vessel map, xvii) RGB image, xviii) blood vessel depth, xix) blood vessel diameter, xx) melanin contrast, xxi) melanin depth, xxii) pigment depth, and xxiii) hair mask file.

19. The method of claim 11, further comprising:
providing at least one first polarizer element disposed in front of and coaxial with an optical axis of the sources, configured to polarize the illumination light from the one or more sources of illumination light at a first polarization; and
providing at least one second polarizer element disposed in front of and coaxial with an optical axis of the one or more sensors, configured to polarize the illumination light reflected from the skin tissue to the one or more sensors in a second polarization, wherein the second polarization is orthogonal to the first polarization.

20. A method for determining a skin treatment regimen, the method comprising:
illuminating a skin tissue with a plurality of illumination light beams having respectively a plurality of light wavelengths;
detecting illumination light reflected from the skin tissue and generating image data;
analyzing the image data and generating skin data indicative of skin optical or physical properties up to 5 millimeters deep; and
analyzing the skin data to determine the skin treatment regimen.
